(19) United States
(12) Patent Application Publication
(10) Pub. No.: US 2005/0016546 A1
(43) Pub. Date: Jan. 27, 2005

(54) Prophylactic device including a liner body and method of use

(76) Inventor: Frederick Pohlman, Jackson Heights, NY (US)

Correspondence Address: LAW OFFICES OF ERIC KARICH, 2807 ST. MARK DR., MANSFIELD, TX, 76063, US

(21) Appl. No.: 10/788,693

(22) Filed: Feb. 27, 2004

Related U.S. Application Data
(60) Provisional application No. 60/490,067, filed on Jul. 26, 2003.
(60) Provisional application No. 60/490,069, filed on Jul. 26, 2003.
(60) Provisional application No. 60/490,076, filed on Jul. 26, 2003.

(51) Int. Cl. A61F 6/06; A61F 6/04

(52) U.S. Cl. 128/844

(57) ABSTRACT

A sheathing device is disclosed for use with a condom to form a prophylactic device to separate a male penis from a female vagina. The sheathing device includes: (i) a liner body formed substantially of a textile material and having an open end, a closed end, and a generally tubular shape adapted to receive the penis, (ii) an inner lubricant layer substantially covering the closed end of the liner body, and (iii) a means for anchoring the open end outside the female vagina. A prophylactic device is described including the liner body and a liquid impervious barrier positioned around the liner body. A method for using the prophylactic device is also described.

### CROSS-REFERENCE TO RELATED APPLICATIONS

This application for a utility patent claims the benefit of the following U.S. Provisional patent applications: Provisional Application Nos. 60/490,067, 60/490,069, and 60/490,076, all filed Jul. 26, 2003. The previous applications are hereby incorporated by reference in their entirety.

### STATEMENT REGARDING FEDERALLY SPONSORED RESEARCH

Not Applicable

### BACKGROUND OF THE INVENTION

1. Field of the Invention

This invention relates generally to prophylactic devices for inhibiting the transmission of contagious diseases during sexual intercourse, and more particularly to a prophylactic device having a liner that enhances the pleasure of sexual intercourse.

2. Description of Related Art

Sexually transmitted diseases include gonorrhea, syphilis, and acquired immunodeficiency syndrome (AIDS), among many others. Prophylactic devices are used to inhibit the transmission of contagious diseases during sexual intercourse. As antibiotic-resistant strains of sexually transmitted diseases are on the rise, and there is no known cure for AIDS, the need for effective prophylactic devices is high. Prophylactic devices are also used to prevent pregnancy.

Condoms are well known prophylactic devices. A typical condom includes a latex rubber sheath for encompassing the male penis. A problem arises with the typical condom in that the required thickness of the latex rubber sheath to avoid rupture during sexual intercourse reduces nerve stimulation in the male penis, thereby reducing male sexual pleasure. As a result, some men shun the use of condoms.

It would be beneficial to have a prophylactic device wherein nerve stimulation in the male penis is not substantially reduced. Such a device would not only increase sexual pleasure, it would also increase the use of condoms and thereby reduce the transmission of sexual diseases and also reduce the number of unwanted pregnancies.

### SUMMARY OF THE INVENTION

A sheathing device is disclosed for use with a condom to form a prophylactic device to separate a male penis from a female vagina. The sheathing device includes: (i) a liner body formed substantially of a textile material and having an open end, a closed end, and a generally tubular shape adapted to receive the penis, (ii) an inner lubricant layer substantially covering the closed end of the liner body, and (iii) a means for anchoring the open end outside the female vagina. A prophylactic device is described including the liner body and a liquid impervious barrier positioned around the liner body. A method for using the prophylactic device is also described.

Other features and advantages of the present invention will become apparent from the following more detailed description, taken in conjunction with the accompanying drawings, which illustrate, by way of example, the principles of the invention.

### BRIEF DESCRIPTION OF THE DRAWING

The accompanying drawings illustrate the present invention. In such drawings:

FIG. 1 is a perspective view of one embodiment of a prophylactic device for separating a male penis from a female vagina during sexual intercourse;

FIG. 2 is a cross-sectional view of the prophylactic device of FIG. 1;

FIG. 3 is a cross-sectional view of one embodiment of a sheathing device for use with a standard condom to form an alternative embodiment of the prophylactic device;

FIG. 4A is a cross-sectional view of one embodiment of a ring shown in FIGS. 2 and 3;

FIG. 4B is a cross-sectional view of another embodiment of the ring of FIGS. 2 and 3;

FIG. 5 is a view of another embodiment of the prophylactic device of FIGS. 1 and 2;

FIG. 6 is a view of a portion of another embodiment of the prophylactic device of FIG. 5, wherein a liquid impervious barrier layer of the prophylactic device includes a venting mechanism for venting air trapped within the prophylactic device during use;

FIG. 7 is a cross-sectional view of the prophylactic device of FIG. 6 as indicated in FIG. 6;

FIG. 8 is a close up cross-sectional view of the prophylactic device shown in FIG. 2, illustrating an inner lubricant layer disposed within the prophylactic device; and

FIG. 9 is a close up cross-sectional view of another alternative embodiment of the prophylactic device, illustrating an outer lubricant layer disposed outside the prophylactic device.

### DETAILED DESCRIPTION OF THE INVENTION

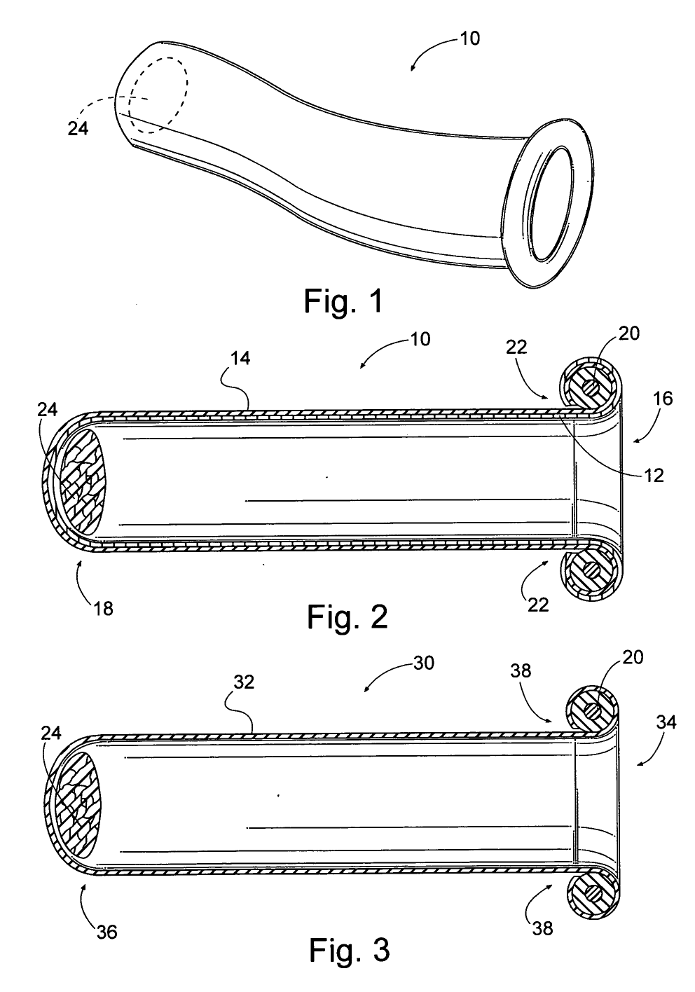

FIG. 1 is a perspective view of one embodiment of a prophylactic device 10 for separating a male penis from a female vagina (not shown) during sexual intercourse. FIG. 2 is a cross-sectional view of the prophylactic device 10 of FIG. 1. In the embodiment of FIGS. 1 and 2, the prophylactic device 10 includes a liner body 12 and a liquid impervious barrier 14 positioned about the liner body 12. The liner body 12 is adapted for encompassing the male penis, has a generally tubular shape, an open end 16, and a closed end 18. The liquid impervious barrier 14 positioned about the liner body 12 also has a generally tubular shape, an open end corresponding to the open end 16 of the liner body 12, and a closed end covering the closed end 18 of the liner body 12.

As described in more detail below, during sexual intercourse, the prophylactic device 10 is designed to stay in a fixed position with respect to the female vagina while the male penis moves with respect to the prophylactic device 10 (and the female vagina). As a result of movement of the male penis with respect to the prophylactic device 10 during sexual intercourse, use of the prophylactic device 10 does not result in reduced nerve stimulation in the male penis as is the case with other prophylactic devices such as condoms. Accordingly, male sexual pleasure is not reduced as it is other prophylactic devices such as condoms.

The liner body 12 is made substantially of a textile material. The textile material may include, for example, elastic textile fibers such as spandex, natural textile fibers such as cotton and silk, synthetic textile fibers, or blends of natural and synthetic textile fibers. One suitable spandex material is Lycra® (E. I. duPont de Nemours & Co., Wilmington, Del.). The textile fibers are preferably knitted or woven to form a fabric, and may be elastic in any dimension (including but not limited to 2-way, 4-way, and multidirectional). During sexual intercourse, the textile material of the liner body 12 provides a soft surface for sliding contact between the male penis and the prophylactic device 10.

In general, the liner body 12 can be made of any textile material, elastic or non-elastic, woven or non-woven, that has at least one relatively smooth surface and is biologically safe. In a preferred embodiment, the liner body 12 is made from elastic fibers (e.g., a spandex material such as Lycra®) woven together to create a pattern and texture commonly referred to as “charmeuse.” In general, charmeuse is a lightweight satin weave fabric with a dull crepe backing. The textile material used in pantyhose, including nylon or a nylon-spandex blend, is also suitable.

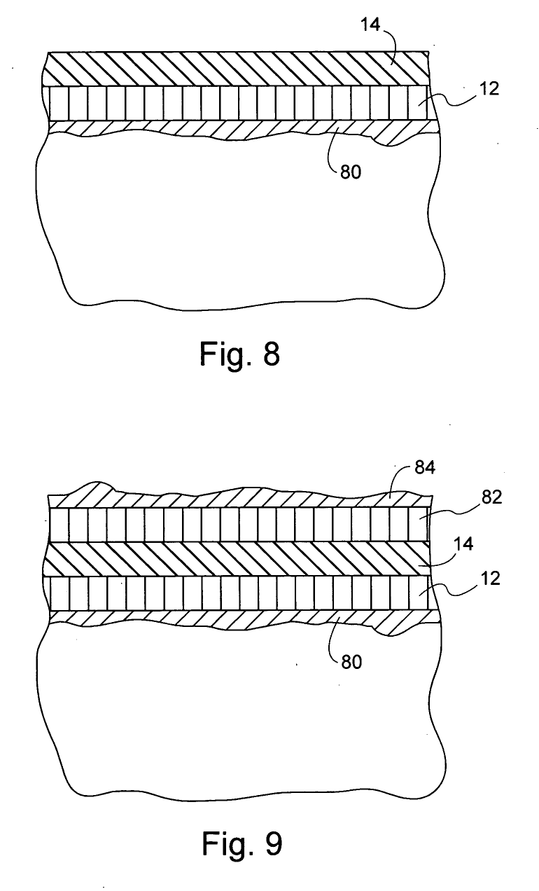

During sexual intercourse, the prophylactic device 10 remains within the female vagina, and the male penis moves or slides against the liner body 12. As shown in FIG. 8, the liner body 12 is substantially covered with an inner lubricant layer 80 to substantially reduce friction between the male penis and the liner body 12. Wetted with the inner lubricant layer 80, the liner body 12 mimics the natural textures and tensions of the female vagina. It is essential that the closed end 18 of the liner body 12 be substantially covered with the inner lubricant layer 80. It is preferred that the closed end be entirely saturated with the inner lubricant layer 80. In some embodiments, the entire liner body 12 is saturated with the inner lubricant layer 80; however, in some embodiments, the open end 16 may not be covered with the inner lubricant layer 80, but may instead frictionally engage the penis.

In general, the inner lubricant layer 80 may be any lubricant suitable for use with the material of the liner body 12. Suitable lubricants include the popular water-based Astroglide® personal lubricant (BioFilm, Inc., Vista, Calif.) and K-Y® lubricating jelly (Advanced Care Products, Ortho Pharmaceutical Corp., Raritan, N.J.). The fine “tooth” of the liner body 12 is particularly well suited for use with a mucous-like lubricant such as the Astroglide® personal lubricant.

The liquid impervious barrier 14 may be formed, for example, from a low modulus, biologically safe material such as latex rubber, a silicone material, or polyurethane. As shown in FIG. 2, an outer surface of the liner body 12 is in contact with an inner surface of the liquid impervious barrier 14. The outer surface of the liner body 12 may not be attached to the inner surface of the liquid impervious barrier 14. For example, the liner body 12 and the liquid impervious barrier 14 may be formed separately, and the liquid impervious barrier 14 may be simply positioned over the liner body 12. Alternately, the outer surface of the liner body 12 may be attached to the inner surface of the liquid impervious barrier 14. For example, the liner body 12 may be formed first, and the liquid impervious barrier 14 may be formed over the liner body 12 (e.g., by coating via dipping or spraying) such that the outer surface of the liner body 12 is integrally formed with the liquid impervious barrier 14.

During manufacture, the liner body 12 may be placed on a dowel with a smooth side against the dowel, and the liquid impervious barrier 14 may be formed over the liner body 12 by dipping or spraying (or both) the liner body 12 with a liquid impermeable material in a flowing state (i.e., latex rubber, a silicone material, or polyurethane).

The prophylactic device 10 includes a means for anchoring the open end 16 of the liner body 12 outside of the female vagina (not shown). In one embodiment, as shown in FIGS. 1-3, the means for anchoring the open end 16 is a ring 20 positioned at the open end 16 of the liner body 12. The ring 20 is dimensioned to fit around the male penis and not to fit into the female vagina (i.e., not to enter the female vulva). The ring 20 is provided for fixedly positioning (i.e., “anchoring”) the open end 16 of the liner body 12 outside the female vagina (i.e., adjacent to the female vulva). In the embodiment of FIGS. 1 and 2, the open end 16 of the liner body 12 and the corresponding open end of the liquid impervious barrier 14 are folded over the ring 20 such that an outer surface of the liquid impervious barrier 14 is adjacent itself at an annular region 22. In one embodiment, the liquid impervious barrier 14 is attached to itself in the annular region 22 (e.g., via heat bonding or a thin layer of a pressure sensitive adhesive material). In another embodiment, the ring 20 includes an adhesive outer layer (not shown) that causes the liquid impervious barrier 14 to adhere to the ring 20. The ring 20 is described in more detail below.

The prophylactic device 10 includes a means for anchoring the closed end 18 of the liner body 12 inside of the female vagina (not shown). In one embodiment, as shown in FIGS. 1-3, the means for anchoring the closed end 18 includes a resilient body 24 positioned within the closed end 18 of the liner body 12. The resilient body 24 is made of a resilient material such as a sponge rubber or similar material, and is provided to fixedly position (i.e., “anchor”) the closed end 18 of the liner body 12 within the female vagina (near the female cervix). In general, the resilient body 24 is dimensioned to remain in place within the female vagina when the closed end 18 of the liner body 12 is positioned within the female vagina.

In alternative embodiment, the means for anchoring the closed end 18 could be a ring (not shown) or similar anchor attached to the exterior of the prophylactic device 10.

FIG. 3 is a cross-sectional view of a one embodiment of a sheathing device 30 for use with an ordinary condom (not shown) to form a prophylactic device. The sheathing device 30 includes a liner body 32 similar to the liner body 12 of FIGS. 1 and 2. In use, the condom is positioned over the sheathing device 30 to form the prophylactic device. During sexual intercourse, the prophylactic device stays in a fixed position with respect to the female vagina while the male penis moves with respect to the prophylactic device and the female vagina.

The liner body 32 is adapted for encompassing the male penis, has a generally tubular shape, an open end 34, and a closed end 36. Like the liner body 12 of FIGS. 1 and 2 and described above, the liner body 32 is made substantially of a textile material. The textile material may include, for example, elastic textile fibers such as spandex (Lycra®), natural textile fibers such as cotton and silk, synthetic textile fibers, or blends of natural and synthetic textile fibers. While the materials listed are examples of suitable materials, the term “textile material” is specifically defined to include all similar fibers and blends that are similar or equivalent to these materials, further including but not limited to Chiffon, Charmeuse, Poly, satin, or other similar material.

In the embodiment of FIG. 3, the sheathing device 30 includes the same ring 20 shown in FIG. 2 and described above. The ring 20 is positioned at the open end 34 of the liner body 32. The ring 20 is provided for fixedly positioning the open end 34 of the liner body 32 outside the female vagina (i.e., adjacent to the female vulva). In the embodiment of FIG. 3, the open end 34 of the liner body 32 is folded over the ring 20 such that an outer surface of the liner body 32 contacts itself in an annular region 38. The outer surface of the liner body 32 is attached to itself in the annular region 38 (e.g., via heat bonding or a thin layer of a pressure sensitive adhesive material).

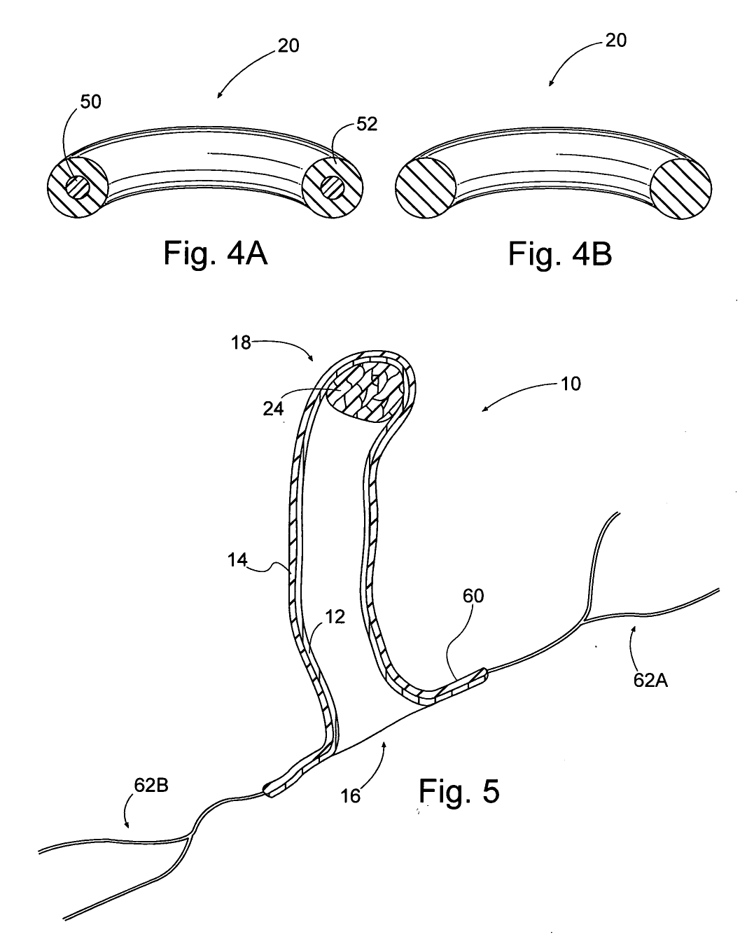

FIG. 4A is a cross-sectional view of one embodiment of the ring 20 of FIGS. 2 and 3. In general, the ring 20 is rigid enough to hold shape, yet soft enough not to cause trauma to the outer portion of the female vagina (i.e., the female vulva). In the embodiment of FIG. 4A, the ring 20 comprises an inner portion 50 and an outer portion 52 surrounding the inner portion 50. The inner portion 50 may, for example, be formed from a harder material (e.g., a rigid plastic). The outer portion 52 may be formed from a soft, rubbery material. Suitable soft, rubbery materials include low modulus materials such as latex rubber and neoprene rubber.

FIG. 4B is a cross-sectional view of another embodiment of the ring 20 of FIGS. 2 and 3. In the embodiment of FIG. 4B, the ring 20 is formed from a material having a hardness between those of the portions 50 and 52 of FIG. 4A, such as, for example, neoprene.

In either embodiment, an outer surface of the ring 20 may be coated with a tacky, non-slip material such as a silicone material or elastic to aid in the folding of layers over the ring 20. Alternately, the outer surface of the ring 20 may be coated with an adhesive material (e.g., a pressure sensitive adhesive).

FIG. 5 is a view of another embodiment of the prophylactic device 10, wherein the means for anchoring includes a semi-rigid panel 60 attached to the open end 16 of the liner body 12. The semi-rigid panel 60 is made of a liquid impervious material, and thus also serves to prevent fluid from contacting the male and female about the penis and vagina (i.e., vulva), respectively, thereby further acting to prevent STDs. The semi-rigid panel 60 may, for example, be formed from a semi-rigid foam or plastic material.

The semi-rigid panel 60 may also include a set of straps 62A and 62B having ends attached to the semi-rigid panel 60. Free ends of the straps 62A and 62B are to be fastened about the woman's legs to help hold the prophylactic device 10 in place such that the open end 16 of the liner body 12 remains outside the female vagina (i.e., adjacent to the female vulva). Alternative embodiments of this general construction may also be used. For example, the semi-rigid panel 60 may be built into women's underpants (i.e., panties, g-string, etc.) to facilitate the comfortable use of the prophylactic device 10.

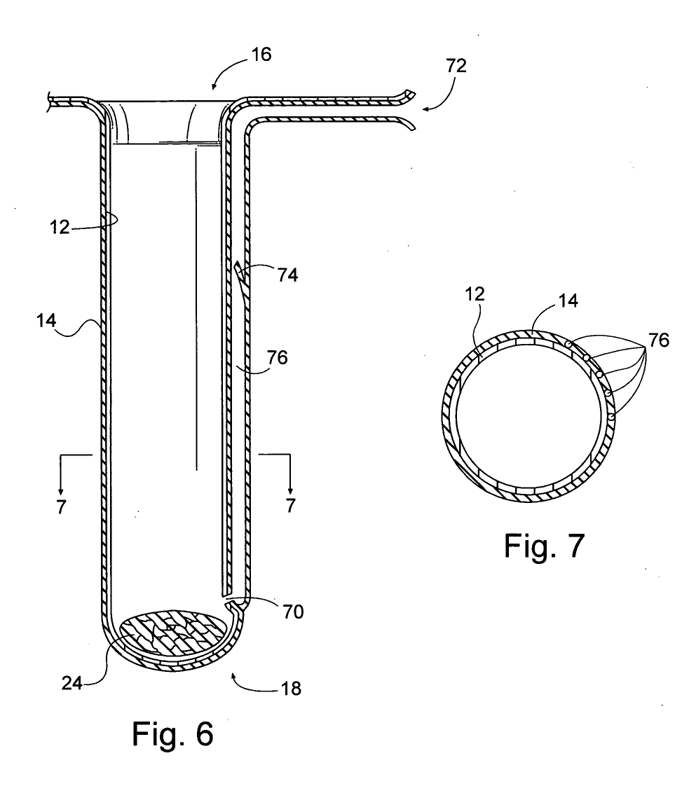

FIG. 6 is a cross section view of another embodiment of the prophylactic device 10 having a venting mechanism for venting air trapped within the prophylactic device 10 during use. The venting mechanism includes vent tubes 76 extending through the liquid impervious barrier layer 14. Each of the vent tubes 76 extends between a first opening 70 at the closed end 18 of the impervious barrier layer 14 and a second opening 72 at the open end 16 of the impervious barrier layer 14. Each of the vent tubes 76 has a one-way valve 74 for letting air trapped within the prophylactic device 10 escape during use. The one-way valves 74 prevent air from entering the prophylactic device 10 via the vent tubes 76.

FIG. 7 is a cross-sectional view of the prophylactic device 10 shown in FIG. 6. As shown in FIG. 7, the vent tubes 76 extend between the closed end 18 and the open end 16 of the liquid impervious barrier layer 14.

A method for using the prophylactic device 10 of FIGS. 1-7 includes providing the liner body 12 described above, (ii) the inner lubricant layer 80, and (iii) the means for anchoring the open end 16 of the liner body 12 outside the vagina. The liner body 12 may be coated with the 14 liquid impervious barrier layer 14, as shown in FIG. 2, or the liner body 12 may be independent of an outer layer, as shown in FIG. 3; however, if the liquid impervious barrier layer 14 is not included, the liner body 12 should be used in conjunction with an ordinary condom.

The closed end 18 of the liner body 12, or the entire liner body 12, is substantially cover with the inner lubricant layer 80. The inner lubricant layer 80 may be applied during the manufacture of the liner body 12, or it may be applied separately just prior to use.

The means for anchoring the open end 16 of the liner body 12 outside the vagina may include, for example, the ring 20 of FIGS. 1-3 and 4A-4B. In this situation, engaging the means may involve attaching the ring 20 to the open end 16 of the liner body 12, and fixedly positioning the ring 20 outside the vagina.

Alternately, the means for anchoring the open end 16 outside the vagina may include the pair of straps (e.g., the straps 62A and 62B of FIG. 5) attached to the open end of the liner body and adapted to be positioned around a woman's legs. In this situation, engaging the means may involve positioning the straps around the woman's legs such that the open end of the liner body is fixedly positioned outside the vagina.

Further, the means for anchoring the open end 16 outside the vagina may include a semi-rigid panel (e.g., the semi-rigid panel 60 of FIG. 5) attached to the open end 16 of the liner body 12. In this situation, the engaging may include positioning the panel about the vagina such that the open end 16 of the liner body 12 is fixedly positioned outside the vagina.

Critical to the function of the liner body 12 is an inner lubricant layer 80. As shown in FIG. 8, the inner lubricant layer 80 is disposed within the prophylactic device 10.

As shown in FIG. 9, an outer layer 82 constructed of the same material used to form the liner body 12 may be used to cover (i.e., encompass) the liquid impervious barrier 14, thereby forming a prophylactic device having textile material inner and outer layers and an intermediate liquid impervious barrier. The outer layer 82 includes an outer lubricant layer 84 disposed outside the prophylactic device. Such a prophylactic device provides a more natural texture for the woman as well as the man.

During manufacture of the prophylactic device 10, the liner body 12 may be placed on a dowel (not shown) with the smooth side against the dowel as described above. The liquid impervious barrier 14 may be formed over the liner body 12 as described above by dipping or spraying the liner body 12 with the liquid impermeable material in the flowing state. When the liquid impermeable material has achieved the substantially non-flowing (i.e., somewhat wet) state, an outer textile sheath similar to the liner body 12 may be applied over the somewhat wet outer surface of the liquid impervious barrier 14.

Prior to use for sexual intercourse, the closed end 18 of the liner body 12 may be positioned within the female vagina using fingers or an applicator. In general, the applicator (not shown) may be a soft tubular device between 6 and 7 inches long, and about an inch wide. The applicator may be formed from, for example, a soft foam material such as Styrofoam® (Dow Corning Company, Midland, Mich.).

In the alternative, the liner body 12 may be positioned around the penis in the same manner as an ordinary condom. Once sexual intercourse has been initiated, the penis functions to position the liner body 12 inside the vagina, where the means for anchoring function to hold the liner body 12 in its proper position.

While the invention has been described with reference to at least one preferred embodiment, it is to be clearly understood by those skilled in the art that the invention is not limited thereto. Rather, the scope of the invention is to be interpreted only in conjunction with the appended claims.

1. A sheathing device for use with a condom to form a prophylactic device adapted to separate a penis from a vagina, the sheathing device comprising:
a liner body substantially comprising a textile material and having an open end, a closed end, and a generally tubular shape adapted to receive the penis;
an inner lubricant layer substantially covering the closed end of the liner body; and
a means for anchoring the open end outside the vagina.

2. The sheathing device as recited in claim 1, wherein the textile material comprises elastic textile fibers.

3. The sheathing device as recited in claim 1, wherein the means for anchoring the open end outside the vagina comprises a ring adapted to be attached to the open end of the liner body and dimensioned to fit around the penis but not fit into the vagina.

4. The sheathing device as recited in claim 1, wherein the means for anchoring the open end outside the vagina comprises a pair of straps attached to the open end of the liner body and adapted to be positioned around a woman's legs.

5. The sheathing device as recited in claim 1, wherein the means for anchoring the open end outside the vagina comprises a semi-rigid panel attached to the open end of the liner body.

6. The sheathing device as recited in claim 1, further comprising a means for anchoring the closed end of the liner body within the vagina.

7. The sheathing device as recited in claim 6, wherein the means for anchoring the closed end of the liner body within the vagina comprises a resilient body adapted to fit within the closed end and fixedly position the liner body within the vagina.

8. A prophylactic device, comprising:
a liner body substantially comprising a textile material and having an open end, a closed end, and a generally tubular shape adapted to receive the penis;
an inner lubricant layer substantially covering the closed end of the liner body; and
a liquid impervious barrier positioned around the liner body.

9. The prophylactic device as recited in claim 8, wherein the textile material comprises elastic textile fibers.

10. The prophylactic device as recited in claim 8, wherein the liquid impervious barrier layer is formed integrally with the liner body.

11. The prophylactic device as recited in claim 8, further comprising a means for anchoring the open end outside the vagina.

12. The prophylactic device as recited in claim 11, wherein the means for anchoring the open end outside the vagina comprises a ring adapted to be attached to the open end of the liner body and dimensioned to fit around the penis and not fit into the vagina.

13. The prophylactic device as recited in claim 11, wherein the means for anchoring the open end outside the vagina comprises a pair of straps attached to the open end of the liner body and adapted to be positioned around a woman's legs.

14. The prophylactic device as recited in claim 11, wherein the means for anchoring the open end outside the vagina comprises a semi-rigid panel attached to the open end of the liner body.

15. The prophylactic device as recited in claim 8, further comprising a means for anchoring the closed end of the liner body within the vagina.

16. The prophylactic device as recited in claim 15, wherein the means for anchoring the closed end of the liner body within the vagina comprises a resilient body adapted to fit within the closed end and fixedly position the liner body within the vagina.

17. The prophylactic device as recited in claim 8, wherein the liquid impervious barrier layer comprises a condom.

18. The prophylactic device as recited in claim 8, wherein the liquid impervious barrier layer comprises a venting mechanism comprising a one-way valve for venting air trapped within the prophylactic device during use.

19. A method for using a prophylactic device, comprising:
providing a liner body substantially comprising a textile material and having an open end, a closed end, and a generally tubular shape adapted to receive the penis;
providing a means for anchoring the open end outside the vagina; and
coating the liner body with an inner lubricant layer.

20. The method as recited in claim 19, further comprising the step of engaging the means such that the open end is fixedly positioned outside the vagina.
